(19) United States
(12) Patent Application Publication
(10) Pub. No.: US 2007/0144937 A1
(43) Pub. Date: Jun. 28, 2007

(54) Product package having a tinted display window

(75) Inventor: Heather S. Gilroy, Reading, PA (US)

Correspondence Address: Gosz and Partners LLP, 450 Bedford Street, Lexington, MA, 02420, US

(73) Assignee: Tyco Healthcare Retail Services AG

(21) Appl. No.: 11/443,877

(22) Filed: May 31, 2006

Related U.S. Application Data
(60) Provisional application No. 60/753,115, filed on Dec. 22, 2005.

(51) Int. Cl. B65D 25/54; A61B 19/02; B65D 85/00

(52) U.S. Cl. 206/776; 206/459.5; 206/440

(57) ABSTRACT

A package for absorbent articles having a tinted or colored window is provided. The color of the window is chosen to match some predetermined characteristic of the enclosed absorbent articles. The tinted window allows the consumers to view individually wrapped absorbent articles, to confirm the number of enclosed absorbent articles, and to determine one or more predetermined characteristics of the absorbent articles without opening the package. The tinting also allows a feature on an individual absorbent article to be highlighted by matching or complimenting the tint to a color or graphic already incorporated in the individual absorbent article.

### CROSS-REFERENCE TO RELATED APPLICATIONS

This application claims the benefit of priority from U.S. Provisional Application No. 60/753,115 filed on Dec. 22, 2005, the specification of which is incorporated herein by reference.

### BACKGROUND OF THE INVENTION

The present invention relates to a package for containing disposable absorbent articles. More particularly, the present invention relates to a package with a tinted window display to allow viewing of absorbent articles without opening the package.

Disposable absorbent articles, such as disposable diapers, training pants, sanitary napkins, protective underwear, and the like, are known. Such articles are provided in various sizes, shapes and conformations to meet the individual requirements of consumers. These articles are typically marketed in packages containing multiple articles, either of similar quality or as part of a multi-use kit. The multiple articles may also be individually wrapped or contain a graphic on the outer surface. The packages, typically in the form of flexible plastic bags, are generally printed with graphic and textual material which helps identify and describe the product and also provide additional information useful to the consumer. Even with the information provided on the package the consumers sometimes want to view the contents of the package to ensure that they are buying the desired product. Thus, the incorporation of a window or other clear area has been used in a package to allow the consumer to view the contents of the package without opening the package.

U.S. Pat. No. 6,601,705 describes a package containing a plurality of individually wrapped absorbent articles such as sanitary napkins. The exterior surface of the package as well as that of individual absorbent articles has a distinctive color scheme, which scheme is indicative of a performance characteristic of the article. Because sanitary napkins are used in plethora of sizes, shapes, conformations and absorbency rates, to meet the differing requirements of individual consumers requires the use of as many product wrappers to match the distinctive color scheme on the exterior of the package. This requires the maintenance of multiple inventories of wrappers and ensuring that the articles are being correctly wrapped and placed in the correct package. This requirement can add complexity and cost to the product manufacturing process.

For diapers or training pants, products generally include colorful graphics on the outer surface, which graphics may be merely decorations differentiating products between male or female child, or incorporating features which aid in toilet training the child. For these types of toilet training articles, U.S. Pat. Nos. 6,318,555 and 6,491,165 describe flexible bags which have two windows or clear areas which display the graphics on the exterior of the product and also allow the consumer to see how many products are in the package. While this may minimize printing on the package, this also necessitates that each of the individual products in the package be printed with or have other unique indicia to aid the consumer in choosing the desired product.

What is needed then is a simplified way to allow consumers to view the contents of a closed package, while also alerting the consumer about the desirable performance characteristics of the enclosed absorbent articles.

It is therefore an objective of the present invention to provide a package for absorbent articles that allows consumers to view the contents of the package without opening the package.

It is another objective of the present invention to provide a package that enables consumers to differentiate different absorbent articles for a more knowledgeable purchase decision.

### SUMMARY

In order to achieve the abovementioned objectives, the invention provides a package comprising a flexible bag having an interior space and an exterior surface, and a plurality of absorbent articles disposed within the interior space. The absorbent articles have one or more predetermined characteristics and are individually wrapped in a wrapper. The exterior surface of the package contains information indicative of one or more predetermined characteristics of the absorbent articles. The package also includes at least one tinted window adapted to allow a viewer to determine the one or more predetermined characteristics of the absorbent articles. The color of the at least tinted window is indicative of some performance characteristic, such as absorbency, thickness, size and the like.

In one embodiment, the exterior surface of the package further comprises a colored section having a color that substantially matches the color of the tinted window.

### BRIEF DESCRIPTION OF THE DRAWINGS

These and other features and advantages of the present invention will be appreciated, as they become better understood by reference to the following Detailed Description when considered in connection with the accompanying drawings:

FIG. 1 is the perspective view of a package with a tinted window containing absorbent articles having a first predetermined characteristic;

FIG. 2 is the representative scheme of a package containing ultra thin regular sanitary napkins with wings;

FIG. 3 is the representative scheme of a package containing maxi superlong sanitary napkins with wings; and

FIG. 4 is a front view of a package for a kit containing two different absorbent articles with two different predetermined characteristics.

### DETAILED DESCRIPTION OF THE INVENTION

The invention provides a package comprising a flexible bag having an interior space and an exterior surface and a plurality of absorbent articles disposed within the interior space. The package also includes at least one tinted window adapted to allow a viewer to determine the one or more predetermined characteristics of the absorbent articles.

In the following specification, specific embodiments of the present invention have been described. However, one of ordinary skill in the art will appreciate that various modifications and changes can be made without departing from the scope of the present invention as set forth in the claims below. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims.

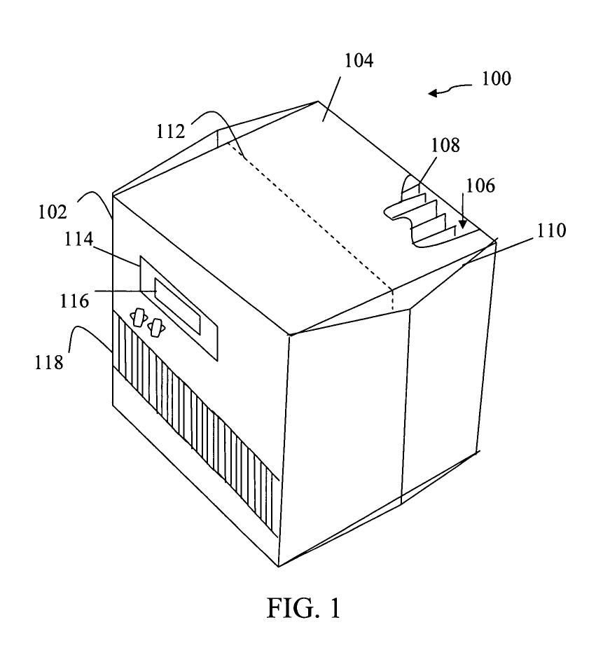

FIG. 1 is the perspective view of a package 100 in accordance with one embodiment of the invention. Package 100 comprises a flexible bag 102 defining an exterior surface 104 and an interior space 106. A plurality of absorbent articles 108 are disposed in interior space 106. Absorbent articles 108 may be sanitary napkins, disposable diapers, training pants, protective underwear and the like. Absorbent articles 108 may be individually wrapped in a wrapper before being placed in package 100.

Referring to FIG. 1, package 100 may also include an inset 110 that functions to strengthen package 100 as well as perforation 112 designed to provide access to the contents of package 100.

Exterior surface 104 has a section 114 containing information indicative of the one or more predetermined characteristics of absorbent articles 108. This information may be provided using a combination of text, graphics and color. In one embodiment of the present invention, the information is provided by means of a specific color 116, which color corresponds to a predetermined characteristic of absorbent articles 108.

Package 100 further comprises a tinted window 118 to allow a consumer to visually inspect the contents of package 100. Tinted window 118 allows the consumer to view the individually wrapped absorbent articles 108 and to determine one or more predetermined characteristics of absorbent articles 108. The one or more predetermined characteristics of absorbent articles 108 may include characteristics like size, shape, thickness and absorbency rates. In one embodiment of the invention, the color of the tinted window is substantially similar to the color 116 indicated in section 114 of package 100.

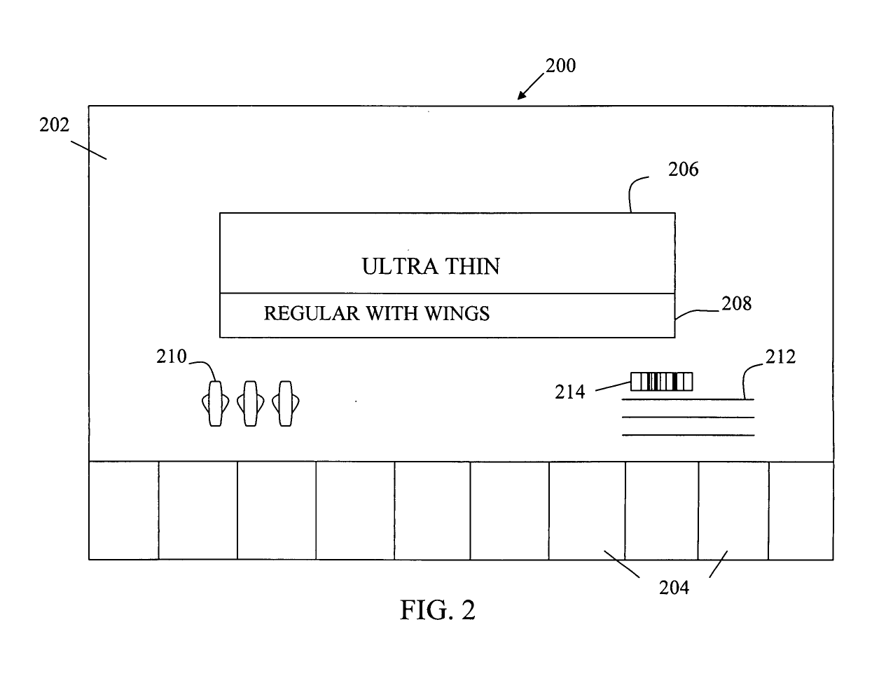

FIG. 2 is a representative example of a package 200 containing ultra thin regular sanitary napkins with wings. As indicated on the exterior surface 202 of package 200, it contains individually wrapped sanitary napkins 204 characterized as “ultra-thin”, having a regular length with wings. This information is provided in section 206 printed on package 200. Section 206 contains the words “Ultra thin-Regular with wings” in the rectangularly shaped area imprinted with a specific color 208, which color 208 corresponds to the regular absorbency characteristic of the enclosed sanitary napkins. For example, color 208 may be red, pink or white to denote respectively the extra, regular and light absorbency of sanitary napkins 204. A graphic 210 may also be included to indicate the size of sanitary napkins. This graphic 210, in the form of the outline of the shape of the enclosed sanitary napkins 204, is also imprinted with the same color 208. Other information such as the seller of the absorbent articles, graphic and other characteristics of sanitary napkins 204 may also be included on exterior surface 202 of package 200 and is generally referred to as “copy”. This information is indicated by lines 212 and design 214. It will be obvious to one skilled in the art that many different kinds of information including, but not limited to, quantity, price, brand name, logo etc. may be included on package 200 on any part of external surface 202.

In order to allow the consumer to view the contents of package 200, a tinted window 216 is provided. Tinted window 216 allows the consumer to view the individually wrapped sanitary napkins 204, to confirm the number of sanitary napkins 204, and to determine one or more predetermined characteristics of the absorbent articles 204. The one or more predetermined characteristics of sanitary napkins 204 may include characteristics like size, shape, thickness and absorbency rates of sanitary napkins 204. In one embodiment of the invention, the color of tinted window 216 is substantially similar to color 208 indicated in section 206 of package 200. Thus, for example, a tint of red color may denote an extra absorbency sanitary napkin, while a tint of pink color may denote a regular absorbency sanitary napkin.

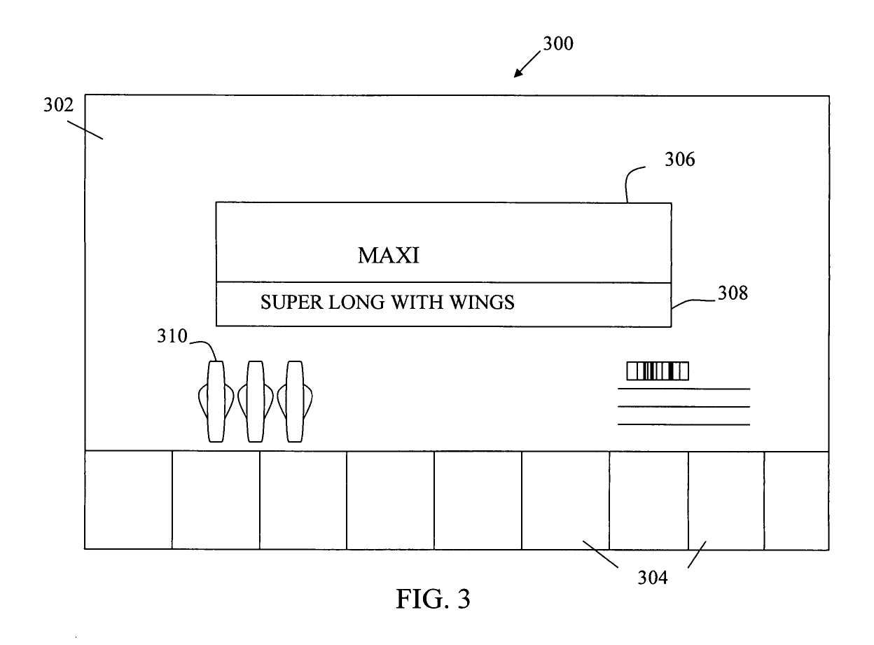

FIG. 3 is another representative example of a package 300 containing nine individually wrapped super long sanitary napkins with wings. As indicated on the exterior surface 302 of package 300, it contains individually wrapped sanitary napkins 304 characterized as “Maxi”, having a superlong length with wings. This information is provided in section 306 printed on package 300. Section 306 contains the words “Maxi-Superlong with wings” in the rectangularly shaped area imprinted with a specific color 308, which color 308 corresponds to the maximum absorbency characteristic of the enclosed sanitary napkins 304. Graphic 310 indicates the size of sanitary napkins 304. Similar to that described above, a tinted window 316 allows the consumer to view the individually wrapped sanitary napkins 304 and to determine one or more predetermined characteristics of absorbent articles 304.

When trying to determine which product to purchase, the consumer need only look at both packages 200 and 300, read the copy, and compare sections 206 and 306 as well as visually inspect sanitary napkins 204 and 304 through tinted windows 216 and 316 to choose the appropriate product. The respective graphics, colored sections and colored tinted windows help the consumers in differentiating the different sanitary napkins without opening the package.

With respect to the individual sanitary napkins, the wrappers may be manufactured of substantially the same color material. In the embodiments shown, the sanitary napkins have a white wrapper. When viewed through the tinted display window 216 and 316, sanitary napkins 204 and 304 appear to have the wrapper of the same color as that of tinted windows 216 and 316. This color may be indicative of some predetermined characteristic of sanitary napkins 204 and 304 described on exterior surfaces 202 and 302 of packages 200 and 300. This simplifies the manufacturing process in that only a single wrapper cover is used rather than having to maintain multiple inventories, while ensuring that the absorbent articles are being correctly wrapped and placed in appropriate packages. The tinting may also allow a feature on an individual absorbent article to be highlighted by matching or complementing the tint to a color or graphic already incorporated in the individual absorbent article.

In order to manufacture packages 200 and 300 for different absorbent articles, the following steps are preferably followed. The packages are surface printed on a flexographic printing press using clear polythene film. The opaque portions of the design are achieved by printing on a base layer of semi opaque white ink. Any design element that is intended to be opaque will have a white back up. The tinted window is achieved by not printing a white base layer in the window portion of the design. The ink, when printed on the clear substrate with no white back-up, results in a tinted window. The printing plates that are used in the printing process are preferably photopolymer plates.

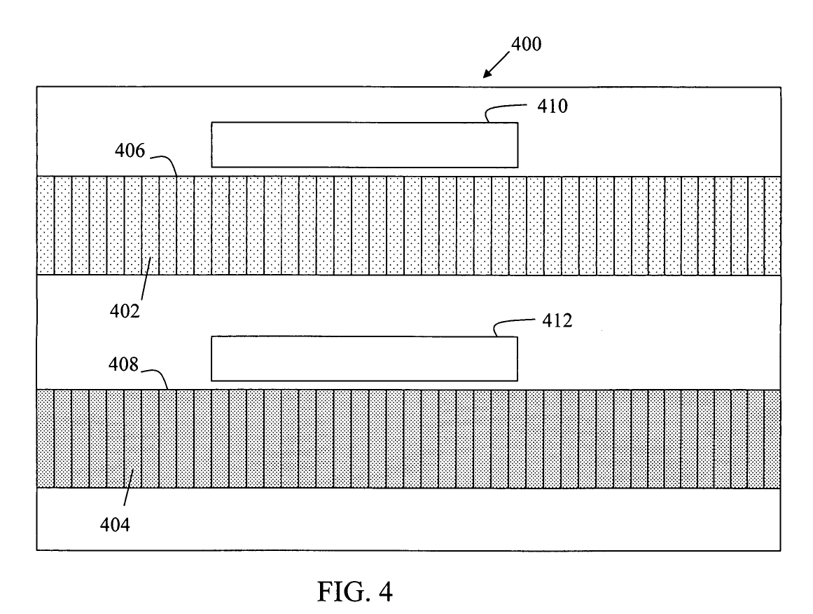

FIG. 4 illustrates a package that uses two differently colored tinted windows to signal absorbent articles with two distinct predetermined characteristics. As shown, package 400 has two sets of absorbent articles 402 and 404 that may have different characteristics. For example, absorbent article 402 may be sanitary napkins with regular absorbency while absorbent article 404 may be sanitary articles with extra absorbency. Tinted windows 406 and 408 have two different colors to signal the different absorbency characteristics of the absorbent articles enclosed in package 400. Further, colored section 410 corresponds to tinted window 406 and has substantially same color as tinted window 406, while colored section 412 corresponds to tinted window 408 and has substantially same color as tinted window 408.

It will be apparent to one skilled in the art that different combinations of colors may be used to denote different predetermined characteristics such as size, shape, thickness and absorbency rates. Further any combination of colors can be used when matching the wrapper color that is perceived through tinted windows 406 and 408 with corresponding colored sections 410 and 412.

The aforementioned embodiments and examples are meant to be just for explanatory purposes only, and are in no way meant to be limiting. Various other configurations are possible within the spirit of the invention. The invention intends to cover all such embodiments within the boundaries defined by the appended claims.

What is claimed is:

1. A package comprising
a flexible bag having an interior space and an exterior surface;
a plurality of absorbent articles disposed within the interior space, the absorbent articles having one or more predetermined characteristics and being individually wrapped in a wrapper;
the exterior surface having information indicative of the one or more predetermined characteristics of the absorbent articles; and
wherein the flexible bag includes at least one tinted window adapted to allow a viewer to determine the one or more predetermined characteristics of the absorbent articles.

2. The package as claimed in claim 1 wherein the one or more predetermined characteristics of the absorbent article is absorbency.

3. The package as claimed in claim 1 wherein the one or more predetermined characteristics of the absorbent article is thickness.

4. The package as claimed in claim 1 wherein the one or more predetermined characteristics of the absorbent article is size.

5. The package as claimed in claim 1 comprising at least two different absorbent articles with distinct predetermined characteristics.

6. The package as claimed in claim 5 comprising at least two differently colored tinted windows allowing the viewer to distinguish between the at least two different absorbent articles.

7. The package as claimed in claim 1 wherein the wrappers are manufactured of substantially the same color material.

8. The package as claimed in claim 1 wherein a feature on the wrapper is highlighted by matching the tint to a feature already incorporated in the wrapper.

9. The package as claimed in claim 1 wherein the exterior surface further comprises a colored section having a color that substantially matches the color of the tinted window.

10. A package for containing absorbent articles, the package comprising:
a) an enclosure defining an interior space and an exterior surface, the interior space containing the absorbent articles;
b) at least one tinted window adapted to allow a viewer to determine one or more characteristics of the absorbent articles; and
c) at least one colored section printed on the exterior surface of the enclosure, the colored section having a color that substantially matches the color of the tinted window.

11. The package as claimed in claim 10 further comprising printed information on the exterior surface of the enclosure, the printed information being indicative of the one or more predetermined characteristics of the absorbent articles.
